(19) United States
(12) Patent Application Publication
(10) Pub. No.: US 2015/0233367 A1
(43) Pub. Date: Aug. 20, 2015

(54) PUMP DEVICE FOR ARTIFICIAL DIALYSIS

(71) Applicant: SHINANO KENSHI CO., LTD., Ueda-shi (JP)

(72) Inventors: Shinsuke SHIMOGATA, Nagano (JP); Masahide TAKAMATSU, Nagano (JP); Kazuya SEKI, Nagano (JP); Naoki KOBAYASHI, Nagano (JP); Keita AIBA, Nagano (JP)

(21) Appl. No.: 14/608,728

(22) Filed: Jan. 29, 2015

(30) Foreign Application Priority Data
Feb. 18, 2014 (JP) 2014-028898
Jan. 8, 2015 (JP) 2015-000973

(51) Int. Cl. F04B 43/12; F04B 17/03; A61M 1/16

(52) U.S. Cl. CPC F04B 43/1253; A61M 1/1621; F04B 17/03

(57) ABSTRACT

A pump device for artificial dialysis includes: a blood pump that transports blood; and a stepping motor that drives the blood pump without using a reduction gear.

### CROSS-REFERENCE TO RELATED APPLICATION

This application is based upon and claims the benefit of priority of the prior Japanese Patent Application No. 2014-028898, filed on Feb. 18, 2014, and the prior Japanese Patent Application No. 2015-000973, filed on Jan. 8, 2015, the entire contents of which are incorporated herein by reference.

### BACKGROUND

(i) Technical Field

The present invention relates to a pump device for artificial dialysis.

(ii) Related Art

Japanese Unexamined Patent Application Publication No. 10-290831 discloses a pump device for artificial dialysis. Such a pump device for the artificial dialysis includes: a blood pump that transports blood; and a direct current brushless motor that drives this blood pump. A reduction gear is provided between the blood pump and the brushless motor. This is because that the blood pump needs to be rotated at a low speed and also at a high torque.

However, when such a reduction gear is used, the driving noise and the vibration might be increased. In particular, the pump device for the artificial dialysis is placed just next to a dialysis patient in order to transport the blood in many cases. Thus, the driving noise and the vibration might be always transmitted to the dialysis patient for several hours during the artificial dialysis, and it might be very stressful.

### SUMMARY

According to an aspect of the present invention, there is provided a pump device, for artificial dialysis, including: a blood pump that transports blood; and a stepping motor that drives the blood pump without using a reduction gear.

### BRIEF DESCRIPTION OF THE DRAWINGS

FIG. 1 is an explanatory view of an artificial dialysis system;

FIG. 2 is an explanatory view of a blood pump;

FIG. 3 is an explanatory view of the blood pump;

FIG. 4 is a block diagram to explain a control system for a stepping motor; and

FIG. 5 is a configuration view illustrating an example of a vector control unit.

### DETAILED DESCRIPTION

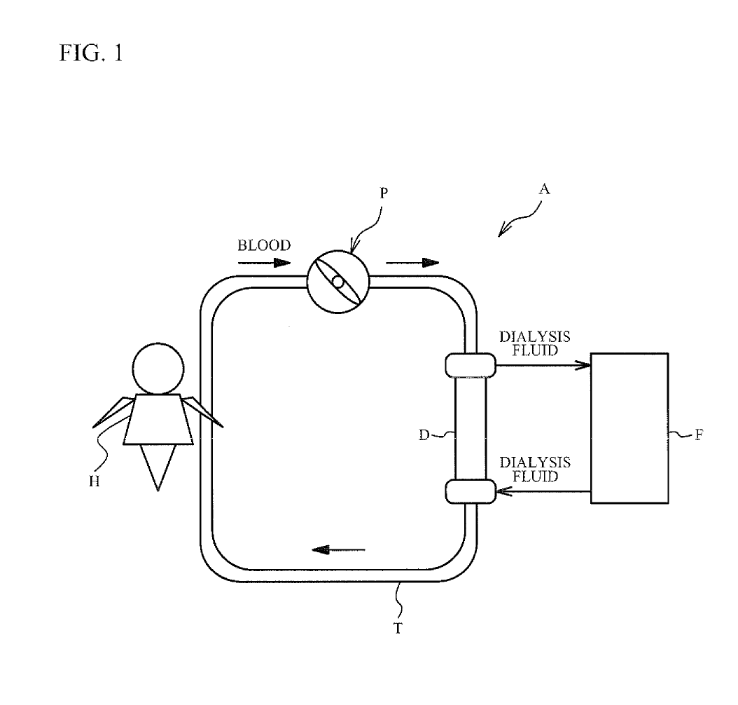

FIG. 1 is an explanatory view of an artificial dialysis system A. Blood of a dialysis patient H is transported through a tube T by a blood pump P. A dialyzer D causes surplus water and waste matter to be discharged from the blood through a semipermeable membrane, and cleans the blood. At this time, a dialysis fluid supply device F supplies a dialysis fluid to the dialyzer D, and the dialysis fluid mixed with internal water and waste matter is transported outside.

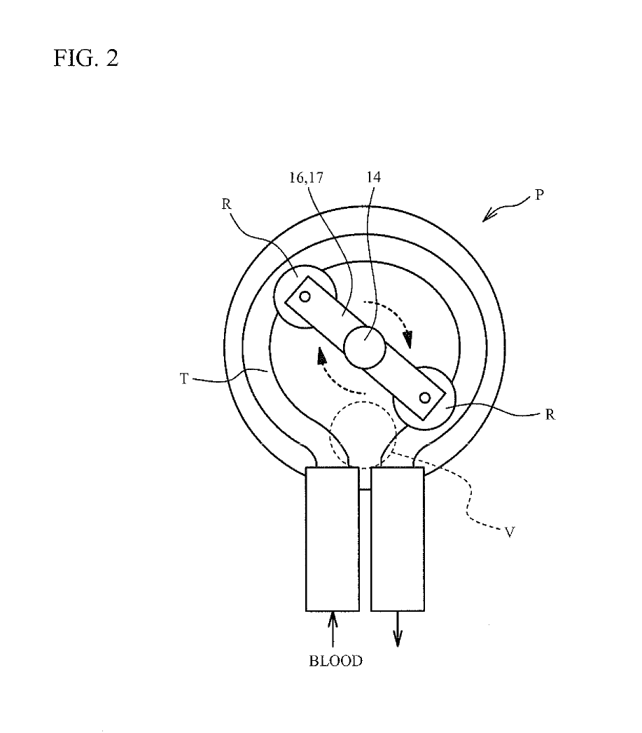

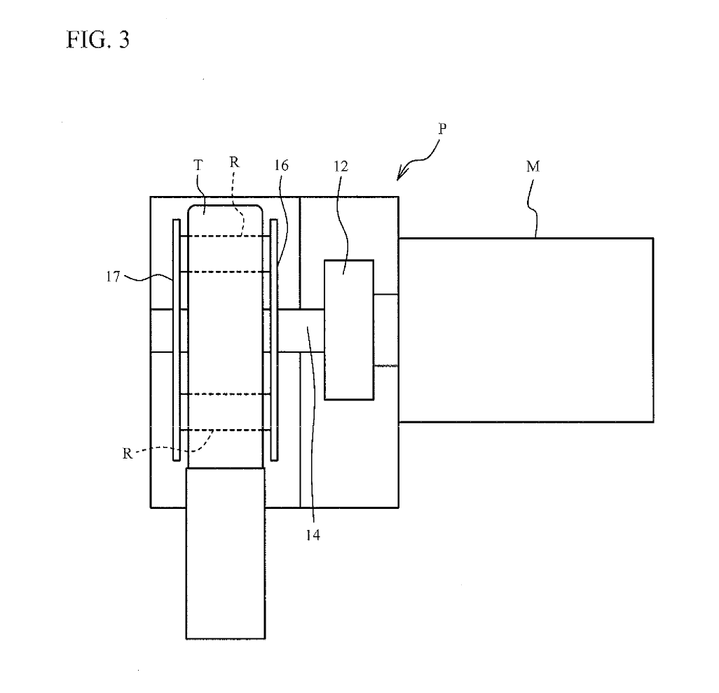

FIGS. 2 and 3 are explanatory views of the blood pump P. A stepping motor M is secured to a rear side of the blood pump P. The blood pump P is driven by the stepping motor M. A device including the blood pump P and the stepping motor M is an example of a pump device for artificial dialysis. An output shaft of the stepping motor M is connected to a rotational shaft 14 of the blood pump P through a coupling member 12. A front end side of the rotational shaft 14 is fitted into and secured to support members 16 and 17 facing each other. Two rollers R are rotatably supported between the support members 16 and 17. When the stepping motor M rotates, the rotational shaft 14, the support members 16 and 17, and the rollers R rotate. The rotational shaft 14 and the support members 16 and 17 are an example of a rotational member rotated by the stepping motor M. The rollers R rotate while pushing an inner side of a part, curved in substantially a U-shape, of the tube T. Thus, the blood can be transported in the direction. Additionally, the rotational speed of the stepping motor M is about from 1 to 110 rpm, but is not limited to this.

The stepping motor M is directly connected to the rotational shaft 14 of the blood pump P without using a reduction gear. That is, the output shaft of the motor M and the rotational shaft 14 of the blood pump P rotate at the same speed. Since the reduction gear is not provided in the pump device for the artificial dialysis according to the present embodiment, the driving noise and the vibration are suppressed, as compared with a case where the reduction gear is provided. Also, the reduction in cost, size, and weight are achieved.

However, even when the reduction gear is not used, the driving noise and the vibration might occur due to another factor. This will be described below. In the blood pump P, there is a space V serving as a region where the rollers R do not push the tube T. When one of the rollers R reaches this space V, only the other roller R pushes the tube T. After one of the rollers R continues rotating from this state and moves away from the space V, both of the two rollers R push the tube T again. In such a way, the rollers R repeatedly come into and out of contact with the tube T. When one of the rollers R reaches the space V and comes out of contact with the tube T, and when one of the rollers R comes into contact with the tube T again, the load applied to the stepping motor M might be changed. For this reason, the pushing force and the pushing amount against the tube T might be changed, so the pushing of the tube T might be too much or not enough. This might cause the driving noise and the vibration to generate from the stepping motor M or the rollers R. Thus, the vector control to be described later is performed in the present embodiment.

Also, a brushless motor is not employed as a motor driving the blood pump P, but the stepping motor M which tends to have characteristics of a low speed and a high torque is employed. This can ensure the rotational torque. It is thus possible to compensate the reduction in the torque due to the elimination of the reduction gear.

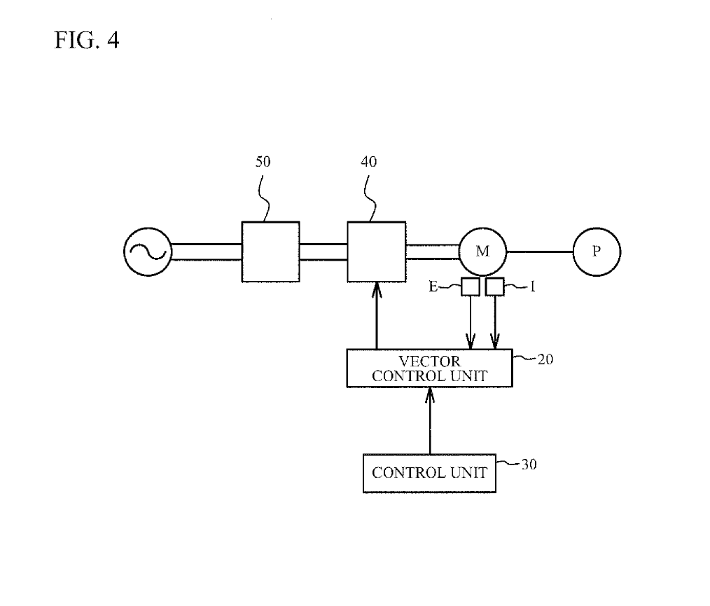

FIG. 4 is a block diagram to explain a control system for the stepping motor M. The stepping motor M is equipped with an encoder E and a current sensor I. The encoder E detects a rotational angular position of a rotor of the stepping motor M. The current sensor I detects a value of current flowing through each phase (an A phase and a B phase) of the stepping motor M. The stepping motor M is vector-controlled based on detection signals which are sent from the encoder E and the current sensor I and which serve as feedback signals.

A converter 50 converts an alternating voltage supplied from an AC power supply into a direct voltage, and supplies the direct voltage to a driver 40. The stepping motor M is supplied through the driver 40 with two-phase power having a predetermined frequency. On the other hand, as for the driver 40, the frequency is controlled by a vector control unit 20. Instructions on speed and rotational direction are input to the vector control unit 20 from a control unit 30 controlling the operation of this blood pump P.

In the driver 40, a voltage type PWM inverter is used. The voltage type PWM inverter is a voltage type inverter using pulse width modulation. In addition, a current type inverter may be used instead of the voltage type inverter.

For example, the driver 40 includes two-phase bridge circuit including eight switching elements, generates driving voltages having two phases of the A and B phases from the input direct current voltage, and supplies the generated two-phase driving voltages to the stepping motor M. ON or OFF states of the switching elements provided in the driver 40 are controlled thereby, so the speed and the torque of the stepping motor M are controlled. As an example, the switching elements of the driver 40 are pulse width modulation (PWM) controlled by the vector control unit 20. Additionally, the switching element is, for example, a field effect transistor (FET), but is not limited to this.

The vector control unit 20 controls a state of intersection of the magnetic flux and the armature current of the stepping motor M by using detection signals sent from the encoder E and the current sensor I, and improves the power factor of the stepping motor M. Therefore, even at a low rotational speed, the output torque of the stepping motor M is controlled to be greater than a torque needed by the blood pump P. An example of the configuration of the vector control unit 20 will be described below.

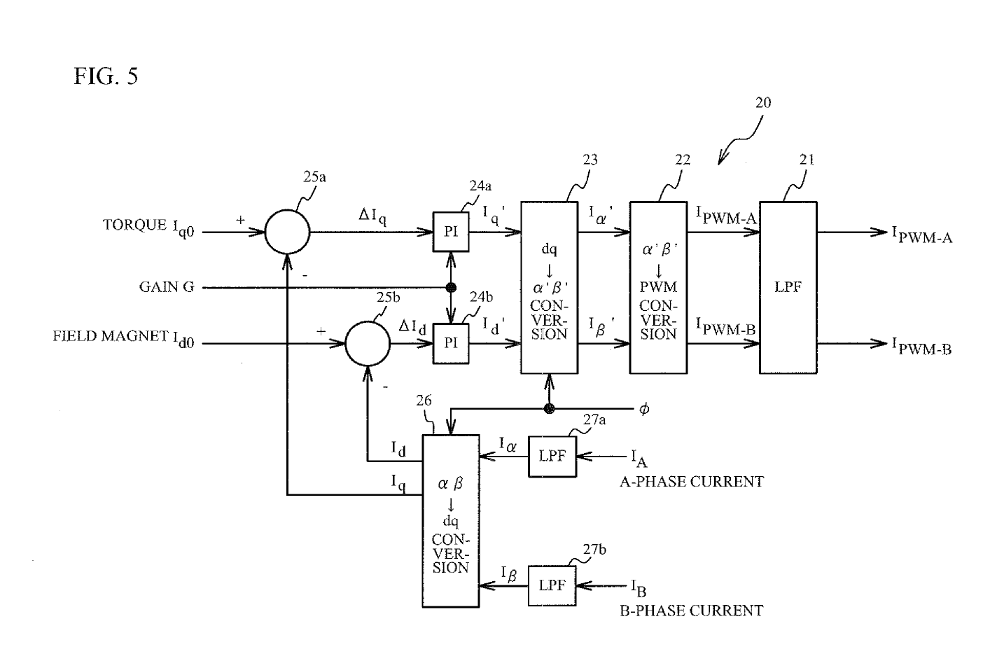

FIG. 5 is a configuration view illustrating an example of the vector control unit 20. The vector control unit 20 includes: a pair of input side low-pass filters (LPFs) 27a and 27b; a first conversion unit 26; and a pair of adders 25a and 25b; a pair of proportional integral (PI) control units 24a and 24b; a second conversion unit 23; a third conversion unit 22; and an output side low-pass filter 21. Current values I_{A} and I_{B} of the A and B phases are input to the pair of input side low-pass filters 27a and 27b, respectively.

After the current values I_{A} and I_{B} of the A and B phases are smoothed by the input side low-pass filters 27a and 27b respectively, two-phase current signals I_{α} and I_{β} of an α-β fixed coordinate system are input to the first conversion unit 26. Also, an angle signal Φ indicating an angle of the rotor detected by the encoder E is input to the first conversion unit 26.

The first conversion unit 26 converts two-phase current signals I_{α} and I_{β} into current signals I_{d} and I_{q} of a d-q coordinate system (d: direct-axis, q: quadrature-axis). The first conversion unit 26 performs the conversion process by using a known mathematical coordinate conversion means. The first conversion unit 26 outputs the current signals I_{d} and I_{q} of the d and q axes obtained by the conversion process to the pair of adders 25b and 25a, respectively.

The current signal I_{q} of the q axis is input to the adder 25a from the first conversion unit 26, and an instruction signal I_{q0} of the torque of the stepping motor M is input to the adder 25a from the control unit 30. The adder 25a detects a difference in signal value between the current signal I_{q} of the q axis and the instruction signal I_{q0} of the torque, and outputs a differential signal ΔI_{q} indicating the above difference to the PI control unit 24a.

The current signal I_{d} of the d axis is input to the other adder 25b from the first conversion unit 26, and an instruction signal I_{d0} of the field magnet of the stepping motor M is input to the other adder 25b from the control unit 30. The adder 25b detects a difference in signal value between the current signal I_{d} of the d axis and the instruction signal I_{d0} of the field magnet, and outputs a differential signal ΔI_{d} indicating the above difference to the other PI control unit 24b. Additionally, the field magnet instruction signal I_{d0} input to the adder 25b from the control unit 30 is fixed to a zero, because an induced voltage of the stepping motor M is much lower than the power supply voltage.

The differential signals ΔI_{q} and ΔI_{d} of the d and q axes are input to the PI control units 24a and 24b from the adders 25a and 25b respectively, and a gain signal G indicating a gain of the PI control is input to the PI control units 24a and 24b from the control unit 30. The PI control units 24a and 24b perform the PI control based on signal values of the differential signals ΔI_{q} and ΔI_{d} and a gain G of the gain signal. The PI control units 24a and 24b respectively generate current control signals I_{d}′ and I_{q}′ of the d and q axes based on the PI control, and output them to the second conversion unit 23.

The second conversion unit 23 converts the current control signals I_{d}′ and I_{q}′ of the d and q axes into two-phase current signals I_{α}′ and I_{β}′ of the α-β fixed coordinate system. The second conversion unit 23 performs the conversion process by using a known mathematical coordinate conversion means. The second conversion unit 23 outputs current signals I_{α}′ and I_{β}′ of α and β axes obtained by the conversion process to the third conversion unit 22.

The third conversion unit 22 converts the current signals I_{α}′ and I_{β}′ of the α and β axes into PWM control signals I_{PWM-A} and I_{PWM-B} of the A and B phases for the switching elements of the driver 40, respectively. The third conversion unit 22 outputs the PWM control signals I_{PWM-A} and I_{PWM-B} which are proportional to the current signals I_{α}′ and I_{β}′ of the α and β axes, respectively. The third conversion unit 22 outputs the PWM control signals I_{PWM-A} and I_{PWM-B} of the A and B phases obtained by the conversion process to the driver 40 through the low-pass filter 21. These PWM control signals I_{PWM-A} and I_{PWM-B} are examples of control signals of the vector control for the stepping motor M.

In this way, the PWM control signals I_{PWM-A} and I_{PWM-B} are output through the low-pass filter 21. Thus, the PWM control signals I_{PWM-A} and I_{PWM-B} gradually change, as compared with a case where they do not pass through the low-pass filter 21. Namely, the low-pass filter 21 which performs the gradual change process for the PWM control signals I_{PWM-A} and I_{PWM-B} is provided in an output stage of the vector control unit 20.

Thus, a torque change of the stepping motor M is reduced. For this reason, the vibration and the noise generated from the stepping motor M are suppressed. As a result, the pump device for the artificial dialysis according to the embodiment reduces the stress of the dialysis patient H.

Since the stepping motor M is for artificial dialysis in the present embodiment, it is sufficient to rotate at a low speed. For this reason, the control system for the stepping motor M can be provided with the low-pass filter 21. In contrast, in a case of a motor which rotates within a rotational speed range between a low speed and a high speed, if the above low-pass filter 21 is provided in a control system for the motor, there is a disadvantage that it is not possible to respond to the high speed rotation.

Additionally, the vector control may be not only the feedback control needing the input from the encoder E but also sensor-less vector control not using such a position sensor. In the sensor-less control, the rotational position and the rotational speed of the stepping motor M are calculated and estimated based on an induced voltage generated in the coil by the magnetic flux of the rotor of the stepping motor M, and the stepping motor M is controlled based on the comparison result between the estimated value and the set value. Further, the change amount of load applied to the stepping motor M is calculated based on the speed information obtained from the position information detected by the encoder E and the value of the coil current, and the control is performed in light of the change amount. It is therefore possible to quickly respond to the load change. Also, it is possible to quickly respond to the pushing force and the pushing amount against the tube T, so it is possible to suppress the driving noise and the vibration generated from the stepping motor M and the rollers R.

In such a way, the stepping motor M is used as the drive source of the blood pump P to which the load greatly changing is applied, and further, the stepping motor M is vector-controlled. It is therefore possible to drive the blood pump P by high torque without using the reduction gear. Also, as the stepping motor M being vector-controlled, it is possible to flexibly respond to the load change and to reduce the power consumption. This means that battery consumption is reduced at the time of power failure, so it is possible to reduce the burdens of medical facilities which are obligated to install power generation facilities for power failure.

While the exemplary embodiments of the present invention have been illustrated in detail, the present invention is not limited to the above-mentioned embodiments, and other embodiments, variations and modifications may be made without departing from the scope of the present invention.

What is claimed is:

1. A pump device for artificial dialysis, the pump device comprising:
a blood pump that transports blood; and
a stepping motor that drives the blood pump without using a reduction gear.

2. The pump device for the artificial dialysis of claim 1, wherein vector control is performed for the stepping motor.

3. The pump device for the artificial dialysis of claim 2, further comprising a low-pass filter,
wherein a control signal of the vector control performed for the stepping motor is output through the low-pass filter.

4. The pump device for the artificial dialysis of claim 1, wherein
the blood pump includes:
a rotational member rotated by the stepping motor; and
a roller provided in the rotational member and pushing a tube through which the blood flows,
the roller repeatedly comes into and out of contact with the tube in response to rotation of the rotational member.
